(19) United States
(12) Patent Application Publication
(10) Pub. No.: US 2009/0240256 A1
(43) Pub. Date: Sep. 24, 2009

(54) Method And Apparatus For Implanting an Augment

(75) Inventor: Aaron P. Smith, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 12/051,174

(22) Filed: Mar. 19, 2008

(51) Int. Cl. A61B 17/58

(52) U.S. Cl. 606/100; 606/99

(57) ABSTRACT

A tool for implanting an augment into a cavity in bone can include a proximal portion and a distal portion. The proximal portion can include a handle. The distal portion can include an adjustment member and an augment interface portion. A shaft can be interconnected between the proximal and distal portions. An engagement portion can be configured at the augment interface portion and coupled to the adjustment member. Movement of the adjustment member can urge the engagement portion in a direction outboard relative to the shaft. A related method can include securing an augment to the tool by locating an augment interface portion of the tool at an engagement site defined on the augment. The adjustment member can be moved on the tool in a first direction, thereby urging an engagement portion of the tool into secured engagement with the engagement site of the augment.

### FIELD

This disclosure relates generally to a method and apparatus for use in orthopedic surgery and, more particular, to a method and apparatus for selectively securing an augment to a tool.

### BACKGROUND

A natural hip joint may undergo degenerative changes due to a variety of etiologies. When such degenerative changes become so far advanced and irreversible, it may ultimately become necessary to replace a natural hip joint with a prosthetic hip. If the acetabulum needs repair, all remnants of articular cartilage may be removed from the acetabulum and an acetabular prosthesis that will accommodate the head or ball of the hip prosthesis may be affixed to the acetabulum.

In some instances, it may also be necessary to remove a defect located adjacent to the acetabulum. It such cases, it may also be necessary to fill the space created by the removed defect with an augment. Sometimes it may be difficult to accurately locate the augment relative to the acetabulum and/or acetabular prosthesis.

### SUMMARY

A tool for implanting an augment into a cavity in bone can include a proximal portion and a distal portion. The proximal portion can include a handle. The distal portion can include an adjustment member and an augment interface portion. A shaft can be interconnected between the proximal and distal portions. An engagement portion can be configured at the augment interface portion and coupled to the adjustment member. Movement of the adjustment member can urge the engagement portion in a direction outboard relative to the shaft.

According to additional features, the first wedge portion can define a first longitudinal axis. The second wedge portion can define a second longitudinal axis. Movement of the adjustment member can urge the first and second wedge portions between an unsecured position wherein the first and second longitudinal axes are collinear and a second position wherein the first and second longitudinal axes are offset.

According to still other features, the adjustment member can comprise a knob having a knurled outer surface. The distal portion can include a cannulated body extending generally between the adjustment member and the augment interface portion. A rod can be slidably disposed through the cannulated body and selectively coupled to the adjustment member and the wedge assembly. A set screw can extend through the cannulated body and engage a flat defined on the rod. The set screw can substantially inhibit rotation of the rod about its longitudinal axis.

A method for repairing a joint socket can include preparing a cavity of the joint socket. A defect can be removed from the joint socket. An augment can be provided having at least a first and a second opening. The augment can be secured to the tool by securing an augment interface portion of the tool at the first opening defined on the augment. The augment can be located at the defect site. A fastener can be passed through the second opening while the tool remains secured to the augment at the first opening.

According to additional features, the method can further include moving first and second wedge portions toward each other causing complementary angled slide surfaces defined on the respective first and second wedge portions to cooperatively engage such that at least one of the first and second wedge portions moves outboard.

Further areas of applicability of the present disclosure will become apparent from the detailed description provided hereinafter. It should be understood that the detailed description and various examples, while indicating various embodiments of the invention, are intended for purposes of illustration only and are not intended to limit the scope of the following claims.

### BRIEF DESCRIPTION OF THE FIGURES

The present invention will become more fully understood from the detailed description and the accompanying drawings, wherein:

FIG. 1 is a side perspective view of a tool for implanting an augment into a bone cavity according to the present teachings;

FIG. 2 is an exploded view of a distal engagement portion of the tool of FIG. 1;

FIG. 3 is a sectional view of the distal engagement portion of the tool shown in an unengaged position and taken along line 3-3 of FIG. 1;

FIG. 4 is a sectional view of the distal engagement portion of the tool shown in an engaged position;

FIG. 5 is a perspective view of an acetabulum shown with the tool secured to an augment and prior to implantation;

FIG. 6 is a perspective view the acetabulum of FIG. 5 shown with the tool locating the augment at a removed defect site;

FIG. 7 is a perspective view of the acetabulum of FIG. 6 shown with the tool removed from the augment and the augment being secured to the cup and acetabulum according to one example;

FIG. 8 is a sectional view of a distal engagement portion of a tool according to additional features and shown in an unengaged position (phantom line) and an engaged position (solid line); and

FIG. 9 is a sectional view of a distal engagement portion of a tool according to additional features and shown in an engaged position.

### DETAILED DESCRIPTION OF VARIOUS EMBODIMENTS

The following description of various embodiment(s) is merely exemplary in nature and is in no way intended to limit the application or uses.

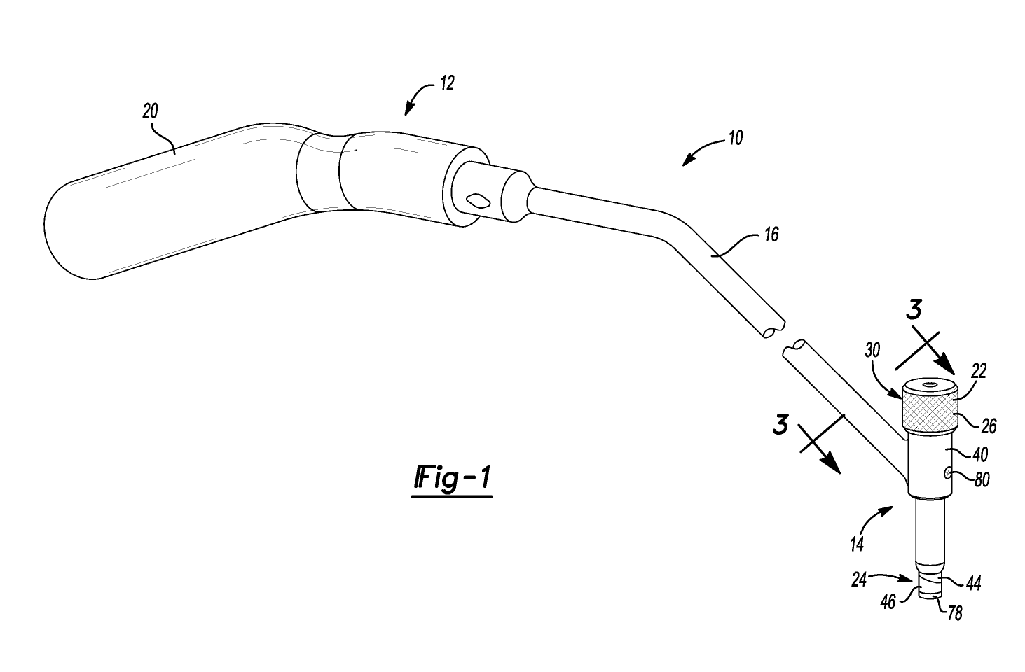

With initial reference to FIG. 1, a tool for implanting an augment at a defect area of a joint socket is shown and generally identified at reference numeral 10. While the following description is directed toward implanting an augment at an acetabular hip socket, it is appreciated that the tool 10 may be used to selectively secure other augments or implants for implantation at other areas of the body.

The tool 10 generally includes a proximal engagement portion 12, a distal engagement portion 14 and a shaft 16 interconnected between the proximal and distal engagement portions 12 and 14, respectively. The proximal engagement portion 12 can define a handle 20. In one example, the shaft 16 can be generally curved between the proximal and distal engagement portions, 12 and 14, respectively.

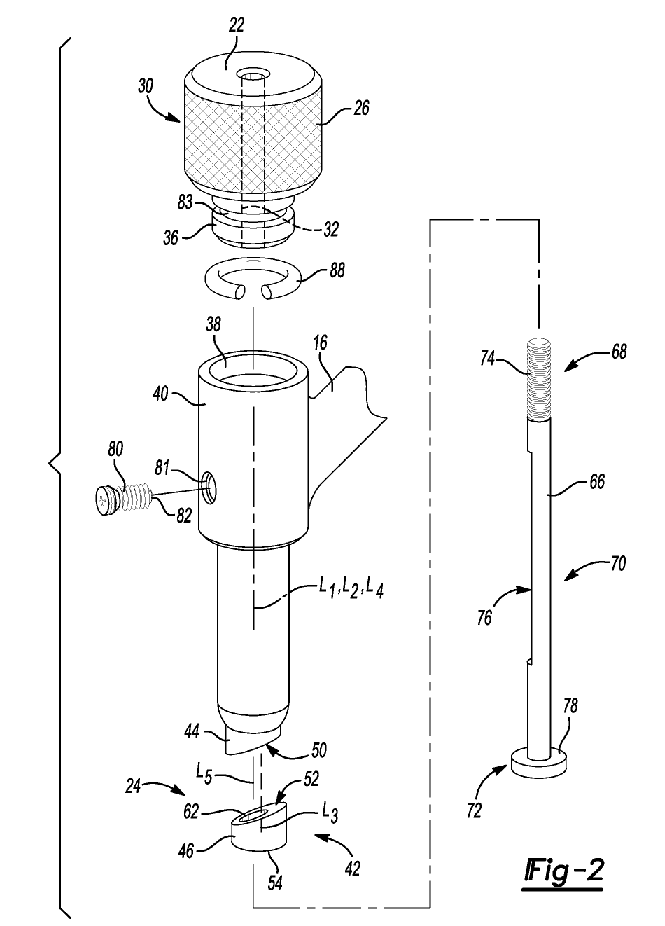

With continued reference to FIG. 1 and additional reference to FIG. 2, the distal engagement portion 14 can define an adjustment member or knob 22 and an augment interface portion 24. The knob 22 can define a generally cylindrical body 26 having a knurled outer surface 30. A threaded bore 32 can be defined at least partially through the knob 22 from a first end. The knob 22 can define a boss 36 that is rotatably received in cylindrical pocket 38 defined in a body 40. The body 40 generally extends between the adjustment member 22 and the augment interface portion 24. The body 40 can generally define a longitudinal axis L₁. The augment interface portion 24 can define a wedge assembly 42 having a first and a second wedge portion 44 and 46, respectively. The first wedge portion 44 can define a first angled surface 50. In one example, the first wedge portion 44 is integrally formed as part of the body 40. The second wedge portion 46 can define a second angled surface 52. In one example, the respective first and second angled surfaces 50 and 52 complement each other such that when engaged to each other, a terminal surface 54 of the second wedge portion 46 is substantially perpendicular to the longitudinal axis L₁ of the body 40.

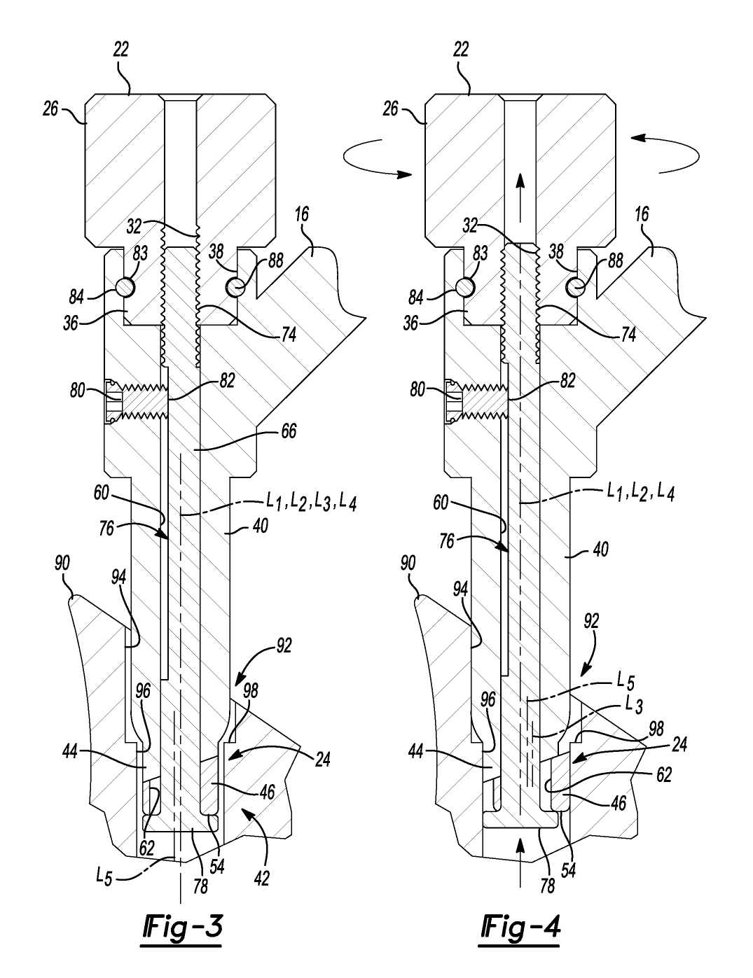

The first wedge portion 44 defines a first longitudinal axis L₂ and the second wedge portion 46 defines a second longitudinal axis L₃. In one example, the longitudinal axis L₁ of the body 40 and the first longitudinal axis L₂ of the first wedge portion 44 are collinear. With additional reference now to FIGS. 3 and 4, a central bore 60 can be defined through the body 40. A longitudinal axis L₄ of the central bore 60 can coincide with the longitudinal axis L₁ of the body 40. An offset bore 62 can be defined longitudinally through the second wedge portion 46. The offset bore 62 can define a longitudinal axis L₅. A diameter of the offset bore 62 can be greater than a diameter of the threaded rod 66.

The distal engagement portion 14 can additionally include a threaded rod 66 slidably disposed through the central bore 60 of the body 40 and the offset bore 62 of the second wedge portion 46. The threaded rod 66 defines a proximal end 68, an intermediate portion 70 and a distal end 72. Threads 74 can be defined at the proximal end 68. The threads 74 can threadably engage the threaded bore 32 formed in the knob 22. A longitudinal flat 76 can be formed along a side of the threaded rod 66 on the intermediate portion 70. A head 78 can be defined on the distal end 72 of the threaded rod 66. In one example, the longitudinal flat 76 can be formed at a location stepped from the threads 74 and the head 78.

A set screw 80 can threadably extend through a passage 81 defined in the body 40. In one example, the set screw 80 can entirely nest within the body 40 in an operating position (FIGS. 3 and 4). A terminal end 82 of the set screw 80 can slidably engage the longitudinal flat 76 of the threaded rod 66. In one example, the set screw 80 can permit axial translation of the threaded rod 66 through the central bore 60 of the body 40 while precluding rotation of the threaded rod 66 about its axis. As will become appreciated from the following discussion, the threaded rod 66 must remain substantially rotationally fixed during rotation of the knob 22. An O-ring 88 can be disposed between a first annular pocket 83 formed around the boss 36 of the knob 22 and a second annular pocket 84 formed around the cylindrical pocket 38 of the body 40.

With specific reference now to FIGS. 3 and 4, use of the tool 10 to securably engage an augment 90 will be described. The augment 90 can define an engagement site 92. The engagement site 92 can define a first and second stepped bore 94 and 96, respectively. An annular ridge 98 can be defined between the first and second stepped bores 94 and 96, respectively. It is appreciated that the augment 90 can define other engagement sites (such as, but not limited to, a uniform bore, a notch, an oblong depression, or other depressions having various geometries).

At the outset, the augment interface portion 24 of the tool 10 is positioned at the engagement site 92. In this particular example, the augment interface portion 24 can be advanced into radial alignment with the second stepped bore 96. Next, the knob 22 may be rotated. Rotation of the knob 22 can cause the thread 74 on the threaded rod 66 to withdraw into the threaded bore 32 of the knob 22. Again, the set screw 80 can slidably communicate along the longitudinal flat 76 to ensure the threaded rod 66 is rotationally fixed about its axis.

Withdrawal of the threaded rod 66 into the knob 22 (e.g., axial movement in a direction upward, as viewed in FIGS. 3 and 4) can cause the head 78 to draw upwardly and against the terminal surface 54 of the second wedge portion 46. As a result, the complementary angled surfaces 50 and 52 of the first and second wedge portions 44 and 46, respectively, can slidably engage. More specifically, the location of the offset bore 62 allows the second wedge portion 46 to be forced outboard and into engagement with the second stepped bore 96 of the augment 90. Because the offset bore 62 has a larger diameter than the threaded rod 66, the second wedge portion 46 can translate in a direction transverse to the longitudinal axis L₁. Concurrently, the first wedge portion 44 can also be forced into engagement with the second stepped bore 96, but at a diametrically opposed and offset location relative to the second wedge portion 46. In one configuration, the body 40 of the tool 10 can be forced against the first stepped bore 94. Those skilled in the art will appreciate that the interaction with the first stepped bore 94 is not necessarily required. Now the augment 90 is secured to the wedge assembly 42 at the augment interface portion 24 of the tool 10. In one example, the surgeon can continue to keep the knob 22 in its rotated position until the augment 90 can be placed in its final destination (i.e., at the defect site). While not shown, a locking feature may be provided to fix the knob 22 at a predetermined rotational position to maintain an outward retention force onto the augment interface portion 24.

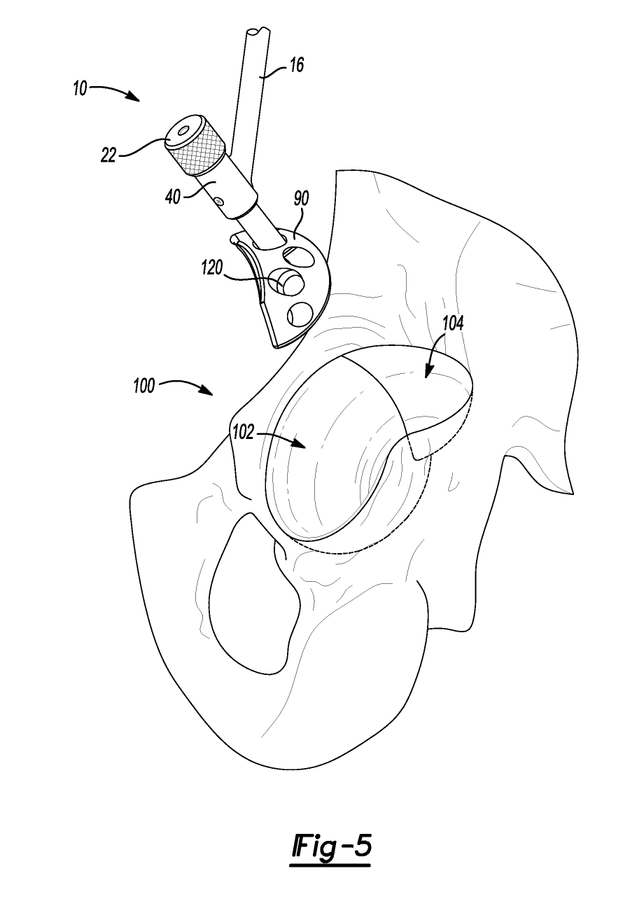

With continued reference to FIGS. 5-7, an exemplary method for using the tool 10 will be described in greater detail. When surgery, such as revision surgery is performed, the primary acetabular cup (not shown) may be removed. An acetabulum 100 may then be reamed, such as with a reamer (not shown). The acetabulum 100 may be generally hemispherically reamed until concentric removal of all acetabular cartilage and/or bone cement is achieved. Once the acetabulum 100 has been appropriately reamed, acetabular trial gauges (not shown), which are well known in the art, may be used to determine and confirm the diameter of the acetabular cup to be used. At this point, a surgeon may access the defect to be reamed and identify an appropriate tool for achieving the proper reaming area. One such tool is disclosed in co-pending application U.S. Ser. No. 11/453,312, filed Jun. 14, 2006, entitled “Method and Apparatus for Reaming an Acetabulum,” also assigned to Biomet, Inc. of Warsaw, Ind., which is incorporated herein by reference. Once the acetabulum 100 has been reamed for receipt of an acetabular cup and the defect has been reamed for receipt of an augment, the acetabulum 100 can define an acetabular socket 102 and a reamed defect site 104.

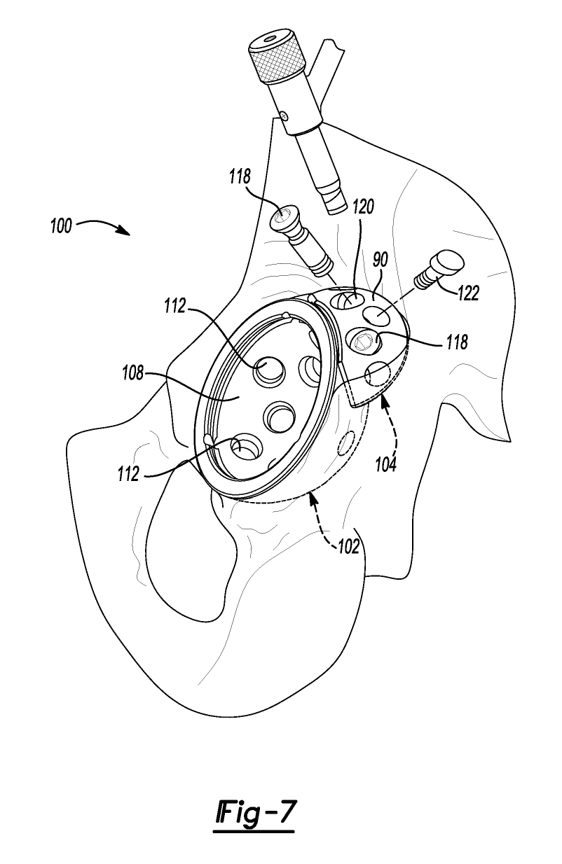

An acetabular cup 108 (FIG. 7) and augment 90 may be implanted. Exemplary acetabular cups and augments may be found in co-pending application U.S. Ser. No. 11/357,868, filed Feb. 17, 2006, entitled “Method and Apparatus for use of Porous Implants,” also assigned to Biomet, Inc. of Warsaw, Ind., which is incorporated herein by reference. In one example, the acetabular cup 108 may be implanted at the acetabular socket 102 and the augment 90 may be implanted at the reamed defect site 104. According to following description, the augment 90 can be implanted into the reamed defect site 104 prior to implanting the acetabular cup 108 into the acetabular socket 102. It is appreciated, however, that the augment 90 may alternatively be implanted subsequent to implantation of the acetabular cup 108.

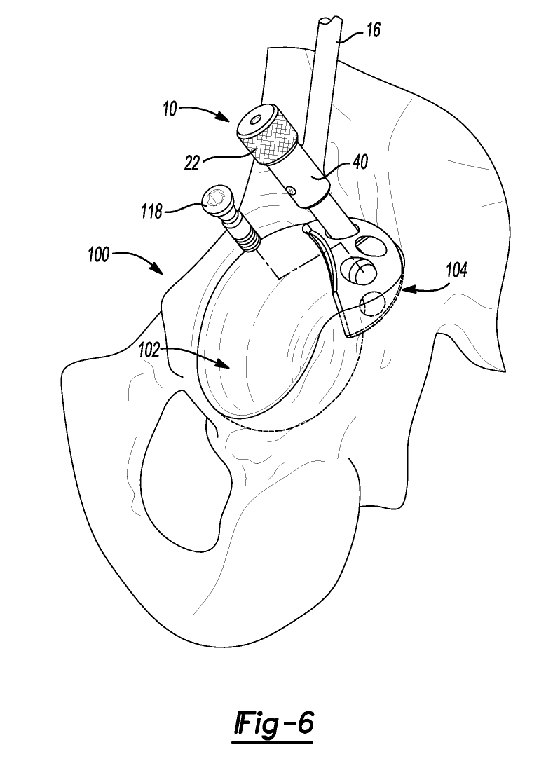

Once the augment 90 has been located at the desired location in the reamed defect site 104 (FIG. 6), the knob 22 may be rotated in a direction that advances the threaded rod 66 away from the knob 22. Upon translation of the threaded rod 66 away from the knob 22, the engagement force of the respective first and second wedge portions 44 and 46 onto the second stepped augment bore 96 is relieved. Explained differently, the respective first and second wedge portions 44 and 46 can move inboard (FIG. 3). The augment interface portion 24 can then be withdrawn from the augment 90.

According to one method of implanting the acetabular cup 108, an impacting instrument (not shown) may be used to properly position the acetabular cup 108. In one example, the impacting instrument may be threadably secured to an apical hole (not shown) of the acetabular cup 108. Once the orientation of the acetabular cup 108 is acceptable, the impacting instrument may be solidly impacted to fully seat the acetabular cup 108 such that firm rim fixation is achieved. Once the acetabular cup 108 has been solidly impacted, the impacting instrument may be carefully removed from the acetabular cup 108.

A plurality of bone screw holes (not specifically shown) may be bored through screw holes 112 in the acetabular cup 108. Once fixation holes have been formed in the acetabulum 100, a depth gauge (not shown), as is also known in the art, may be used to determine the length of the bone screws. With the length of the bone screws determined, a bone screw or multiple screws (not shown) may be inserted into screw holes 112. Other bone screws, such as fixation screws 118, may be similarly used though openings or bores 120 in the augment 90 to secure the augment 90. A coupling screw 122 can also be optionally used to secure the augment 90 to the acetabular cup 108. It is appreciated that other methods may be used to secure the acetabular cup 108 and/or the augment 90. In one example, an adhesive (such as bone cement) may be additionally or alternatively used.

In one advantage of using the tool 10, the augment 90 can be securely retained by utilizing only one of the bores 120. As a result, a user can positively position the augment 90 into a desired position at the defect site 104 with the tool 10 while concurrently passing a fixation screw 118 through any of the other available bores 120. Similarly, the coupling screw 122 can additionally or alternatively be passed through one of the bores 120 while the augment 90 is securely retained (by way of another bore 120) by the tool 10. Furthermore, the slim profile of the distal engagement portion 14 provides a surgeon ample working space and a favorable viewing angle for accessing the remaining bores 120 and manipulating the augment 90 as a whole relative to the defect site 104 during implantation.

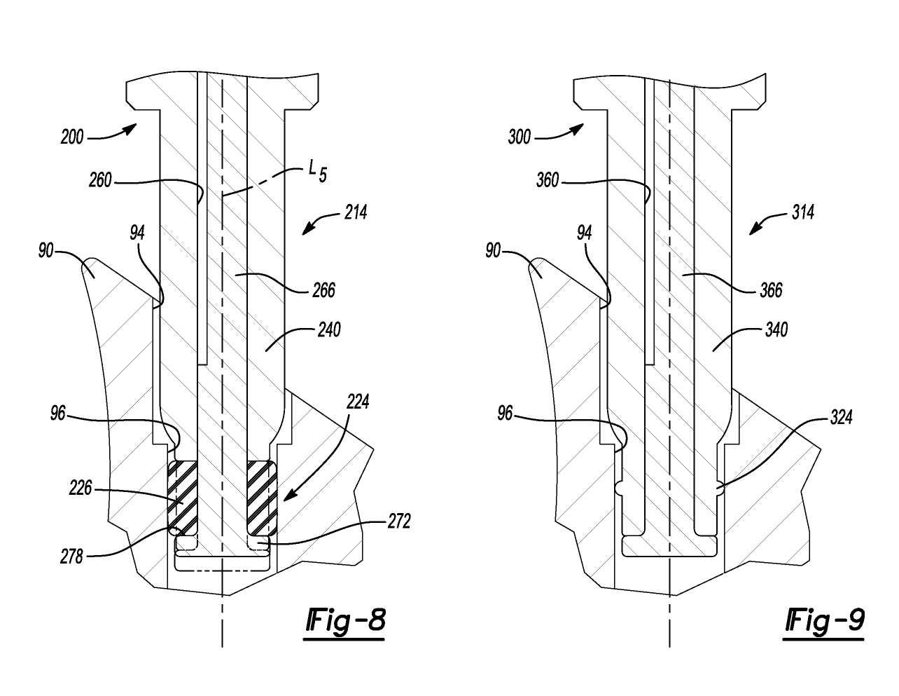

Turning now to FIG. 8, a tool 200 according to additional features will be described. The tool 200 can include a distal engagement portion 214. The tool 200 can also define a proximal engagement portion 12 and a shaft 16, such as described in relation to the tool 10 above. The distal engagement portion 214 can define a body 240 that has an augment interface portion 224. The augment interface portion 224 can define an expanding member 226. The distal engagement portion 214 can additionally include a threaded rod 266 that is slidably disposed through a central bore 260 of the body 240. The threaded rod 266 can be advanced along its axis L₅ by way of a threaded knob, such as described above in relation to the tool 10. The threaded rod 266 can define a head 278 at a distal end 272. The expanding member 226 can be bound on opposite ends by the body 240 and the head 278, respectively. In one example, the expandable member 226 can be formed of an elastomeric material. Advancement of the threaded rod 266 in a direction upward as viewed in FIG. 8 along axis L₅ can cause the expandable member 226 to expand from a first position (phantom line) outwardly to a second position (solid line) into engagement with the bore 96 of the augment 90 to securably retain the augment 90 to the tool 200. Once the augment 90 has been located and optionally secured at the desired location with fixation screws 118 and/or coupling screw 122 (see FIG. 7) in the reamed defect site 104 (see FIG. 6), the threaded rod 266 can be advanced in a direction downward (as viewed in FIG. 8) along the axis L₅. As the threaded rod 266 is advanced downward, the expandable member 226 becomes decompressed and therefore can return to its relaxed state (phantom line). In the relaxed state, the expandable member 226 does not securably engage the bore 96 and the tool 200 can thereafter be removed from the augment 90.

Turning now to FIG. 9, a tool 300 according to additional features is shown. The tool 300 can include a distal engagement portion 314. The tool 300 can also define a proximal engagement portion 12 and a shaft 16, such as described in relation to the tool 10 above. The distal engagement portion 314 can define a body 340 that has an augment interface portion 324. In one example, the augment interface portion 324 can be integrally formed with the body 340. The augment interface portion 324 can generally define a raised radius portion of the body 340. According to one example, the raised radius portion can have an outer dimension that is substantially equivalent to the bore 96 of the augment 90. In this way, the augment interface portion 324 can define an interference fit with the bore 96 of the augment 90. As can be appreciated in the example shown in FIG. 9, the tool 300 does not necessarily require any moving parts. While a shaft 366 is shown through a central bore 360 of the body 340, it can alternatively be integrally formed with the body 340 or be removed entirely. In one example, a surgeon can attach the augment 90 to the tool 300 by inserting the distal engagement portion 314 into the bore 96, thereby creating an interference fit between the augment interface portion 324 and the bore 96. Once the augment 90 has been located at the desired location in the reamed defect site 104 (see FIG. 6), the augment 90 can be held in place by a secondary tool, or by fixation screw 118 (see FIG. 6) and/or by a coupling screw 122 (see FIG. 7). The distal engagement portion 314 can then be withdrawn from the bore 96.

Those skilled in the art can now appreciate from the foregoing description that the broad teachings of the present invention can be implemented in a variety of forms. Therefore, while this invention has been described in connection with particular examples thereof, the true scope of the invention should not be so limited since other modifications will become apparent to the skilled practitioner upon a study of the drawings, the specification and the following claims.

What is claimed is:

1. A tool for implanting an augment into a cavity in bone, the tool comprising:
a proximal portion having a handle;
a distal portion including an adjustment member and an augment interface portion;
a shaft interconnected between the proximal and distal portions; and
an engagement portion configured at the augment interface portion and coupled to the adjustment member, the engagement portion including a cylindrical member having a passage defined therethrough, the passage being offset from a longitudinal centerline of the cylindrical member, wherein movement of the adjustment member urges the engagement portion in a direction outboard relative to the shaft.

2. The tool of claim 1 wherein the engagement portion further comprises:
a first wedge portion defining a first longitudinal axis;
wherein the cylindrical member defines a second wedge portion defining a second longitudinal axis; and
wherein movement of the adjustment member urges the first and second wedge portions between an unsecured position, wherein the first and second longitudinal axes are collinear, and a secured position, wherein the first and second longitudinal axes are offset.

3. The tool of claim 1 wherein the adjustment member comprises a knob.

4. The tool of claim 3 wherein the knob defines a knurled outer surface.

5. The tool of claim 1 wherein the distal portion further comprises a cannulated body extending generally between the adjustment member and the augment interface portion.

6. The tool of claim 5, further comprising:
a rod slidably disposed through the cannulated body and selectively coupled between the adjustment member and the wedge assembly.

7. The tool of claim 6 wherein the rod is threadably connected to the adjustment member.

8. The tool of claim 7, further comprising a set screw wherein:
the rod defines a flat formed along an intermediate portion thereof; and
the set screw extends through the cannulated body, engages the flat and substantially inhibits rotation of the rod about its longitudinal axis.

9. The tool of claim 7 wherein the first and second wedge portions define a first passage and a second passage, respectively, the rod extending through both of the first passage and the second passage.

10. The tool of claim 9 wherein:
the rod defines a head; and
the first and second wedge portions are captured between the cannulated body and the head.

11. The tool of claim 10 wherein the first wedge portion defines a first slide surface defining a first non-orthogonal plane relative to the first longitudinal axis and the second wedge portion defines a second slide surface defining a second non-orthogonal plane relative to the second longitudinal axis, wherein the first and second wedge portions slidably communicate along the first and second slide surfaces upon movement of the adjustment member, wherein the second wedge portion translates in a direction transverse to the first longitudinal axis.

12. A tool for implanting an augment into a cavity in bone, the tool comprising:
a proximal portion having a handle;
a distal portion including an adjustment member and an augment interface portion;
a shaft interconnected between the proximal and distal portions; and
a wedge assembly configured at the augment interface portion and coupled to the adjustment member, the wedge assembly comprising a first wedge portion having a first longitudinal axis and a second wedge portion having a second longitudinal axis,
wherein movement of the adjustment member urges the first and second wedge portions between an unsecured position, wherein the first and second longitudinal axes are collinear, and a secured position, wherein the first and second longitudinal axes are offset.

13. The tool of claim 12 wherein:
the first wedge portion defines a first slide surface defining a first non-orthogonal plane relative to the first longitudinal axis and the second wedge portion defines a second slide surface defining a second non-orthogonal plane relative to the second longitudinal axis; and
wherein the first and second wedge portions slidably communicate along the first and second slide surfaces upon movement of the adjustment member.

14. The tool of claim 12 wherein the distal portion further comprises a cannulated body extending generally between the adjustment member and the augment interface portion.

15. The tool of claim 14, further comprising:
a rod slidably disposed through the cannulated body and selectively coupled between the adjustment member and the wedge assembly.

16. The tool of claim 14, further comprising a set screw wherein:
the rod defines a flat formed along an intermediate portion thereof; and
the set screw extends through the cannulated body, engages the flat and substantially inhibits rotation of the rod about its longitudinal axis.

17. A method for repairing a joint socket the method comprising:
(a) preparing a cavity of the joint socket including removing a defect from a defect site;
(b) providing an augment having at least a first opening;
(c) securing the augment to a tool, the securing comprising:
securing an augment interface portion of the tool at the first opening defined on the augment;
(d) locating the augment at the defect site; and
(e) passing a fastener through the second opening while the tool remains secured to the augment at the first opening.

18. The method of claim 17 wherein providing the augment includes providing an augment having only one opening and wherein securing the augment includes securing the augment interface portion at the only one opening.

19. The method of claim 18 wherein preparing the cavity comprises:
reaming an acetabular cavity with a reamer; and
reaming the defect adjacent to the reamed cavity with the reamer.

20. The method of claim 18 wherein securing the augment to the tool comprises:
moving an adjustment member on the tool a first direction, thereby urging at least one of a first and a second engagement portion of the tool into secured engagement with the opening of the augment.

21. The method of claim 20, further comprising releasing the augment from the tool, wherein the releasing comprises:
moving the adjustment member on the tool a second direction, thereby disengaging the at least one of the first and the second engagement portion from the opening of the augment.

22. The method of claim 18 wherein securing the augment interface portion of the tool comprises:
inserting a longitudinal portion of a distal engagement portion defined on the tool into the opening defined on the augment.

23. The method of claim 18 wherein moving the adjustment member on the tool a first direction includes rotating a knob around an axis.

24. The method of claim 18 wherein securing the augment to the tool comprises:
compressing an expandable member to urge the expandable member radially outward into engagement with the opening.

25. The method of claim 22 wherein securing the augment to the tool comprises:
creating an interference fit between a raised radius portion formed on the longitudinal portion and a bore defined by the opening.

26. The method of claim 21 wherein urging at least one of the first and second engagement portions of the tool comprises:
moving first and second wedge portions toward each other wherein complementary angled slide surfaces defined on the respective first and second wedge portions cooperatively engage such that at least one of the first and second wedge portions moves outboard.

27. The method of claim 26 wherein moving the first and second wedge portions toward each other includes drawing a rod in a direction toward the adjustment member through first and second passages defined by the first and second wedge portions respectively, upon rotation of the adjustment member.
